(19) United States
(12) Patent Application Publication
(10) Pub. No.: US 2016/0008149 A1
(43) Pub. Date: Jan. 14, 2016

(54) INTRAVASCULAR STENT WITH HELICAL STRUTS AND SPECIFIC CROSS-SECTIONAL SHAPES

(71) Applicant: National Taiwan University, Taipei City (TW)

(72) Inventors: Hao-Ming HSIAO, Taipei City (TW); Hsin CHEN, Taipei City (TW)

(21) Appl. No.: 14/558,765

(22) Filed: Dec. 3, 2014

(30) Foreign Application Priority Data
Jul. 11, 2014 (TW) 103123943

(51) Int. Cl. A61F 2/86; A61L 31/02; A61L 31/06; B33Y 80/00

(52) U.S. Cl. CPC A61F 2/86; B33Y 80/00; A61L 31/022; A61L 31/06; A61F 2002/828

(57) ABSTRACT

The present invention relates to a stent, comprising: a plurality of radially-expandable rings arranged along a longitudinal axis, wherein each radially-expandable ring may comprise a plurality of bar arms and a plurality of crowns, and adjacent crowns are connected by the bar arms therebetween; and a plurality of connectors between the radially-expandable rings for connecting such radially-expandable rings; wherein a cross-sectional shape of the bar arms, the crowns or the connectors may comprise helical structures, specific cross-sectional shapes, or a combination thereof. New stent manufacturing techniques, such as the 3D additive printing, could be used for making these proposed stents feasible.

### CROSS REFERENCE TO RELATED APPLICATION

This application claims the benefits of Taiwan Patent Application Serial Number 103123943, filed on Jul. 11, 2014, the subject matter of which is incorporated herein by reference.

### BACKGROUND OF THE INVENTION

1. Field of the Invention

The present invention relates to an intravascular stent. More specifically, the present invention relates to an intravascular stent with helical structures, specific cross-sectional shapes, or any combination thereof prepared by a 3D additive printing method.

2. Description of Related Art

Vascular stent surgery has become a standard treatment for cardiovascular disease. A variety of stent-related inventions, such as drug-eluting stents, bioresorbable stents, and the like, are widely used for treating various vascular diseases, such as coronary artery, peripheral artery, and bile duct diseases.

For the clinical application of the intravascular stent, important factors in stent design are conformability, radial strength, flexibility, deliverability, and fracture or fatigue resistance. Therefore, conventional stents made of metals or alloys have been used in clinical practice due to their good clinical outcome. However, due to their long-term presence in the body, the use of such metallic stents has raised concerns about thrombosis. To alleviate such concerns, stents made of bioresorbable materials have been developed to reduce the problem of thrombosis.

The stress of a stent is mainly concentrated on the crowns or the connectors, while the bar arms bear almost no force. Due to the limitations of stent manufacturing techniques (such as laser cutting), conventional solutions typically re-arrange the stress distribution of an intravascular stent by variations in the design patterns or parameters. However, the changes to the design patterns or parameters of the stent still have their limitations when conventional stent manufacturing techniques are applied. Moreover, in a bioresorbable stent, a great amount of stress tends to concentrate in the crowns and can cause cracks or fractures due to the inherent properties of polymer materials.

3D additive printing is a rapid prototyping technology wherein a product is constructed layer-by-layer of an adhesive material such as a powdered metal or plastic. This method allows rapid prototyping, significantly reduces the consumption of the raw materials, and is customizable. 3D additive printing is considered to have the potential to change future medical industry. One new trend in these developments is the manufacturing of a 3D printed stent. In addition to allowing customization of stents for patients, 3D additive printing can also break through the limitations of traditional stent design, allowing ideas that were unfeasible in the past to be realized. The intravascular stent with regio-selective materials and structures of the present invention is one example thereof.

Therefore, according to clinical needs, it is desirable to provide an intravascular stent with helical structures or specific cross-sectional shapes that has the properties of good conformability, radial strength, flexibility, deliverability, and fracture or fatigue resistance.

### SUMMARY OF THE INVENTION

The object of the present invention is to provide an intravascular stent comprising a plurality of bar arms, a plurality of connectors, and a plurality of crowns, which respectively comprise a helical structure or a specific cross-sectional shape to improve the stent properties such as deliverability. Thus, an intravascular stent with high deliverability, high flexibility, high conformability, and high fracture or fatigue resistance is provided.

To achieve the object described above, the present invention provides a stent comprising a plurality of radially-expandable rings arranged along a longitudinal axis, wherein each of the radially-expandable rings comprises a plurality of bar arms and a plurality of crowns, wherein adjacent crowns are connected by the bar arms therebetween; and a plurality of connectors is disposed between the radially-expandable rings for connecting the radially-expandable rings.

In the stent of the present invention mentioned above, at least one of the bar arms, the crowns, and the connectors may comprise a helical structure. Alternatively, a cross-sectional shape (cross-section) of at least one of the bar arms, the crowns, or the connectors is selected from the group of circular, oval, triangular, rectangular, hexagonal, octagonal, polygonal and airfoil shapes, or any combination thereof.

In the stent of the present invention mentioned above, at least one of the bar arms, the crowns, and the connectors may comprise a helical structure, while a cross-sectional shape (cross-section) of at least one of the bar arms, the crowns, or the connectors is selected from the group of circular, oval, triangular, rectangular, hexagonal, octagonal, polygonal and airfoil shapes, or any combination thereof.

In the stent of the present invention mentioned above, the deliverability of the stent is improved by inserting the helical structures into the structure of the stent. Therefore, a stent with high deliverability, high flexibility, high conformability, and high fracture or fatigue resistance can be provided. Furthermore, the helical structures can be applicable to other types of stents, and said application is not limited to the present invention. Preferably, in the stent of the present invention mentioned above, at least one of the helical structures of the bar arms, the crowns, and the connectors may be a continuous or discontinuous helical structure. A person skilled in the art is capable of applying the continuous or discontinuous helical structures to different components of the stent as needed to improve the properties of the stent, such as deliverability.

Moreover, referring to the stent of the present invention mentioned above, a person skilled in the art may apply the helical structures to an appropriate location in the bar arms, the crowns, the connectors, or any combination thereof according to the properties of the material of the stent, the processing conditions for preparing the stent, the design patterns of the stent, or other design parameters of the stent. Specifically, in one embodiment, the connectors may comprise the helical structures. In another embodiment, the bar arms may comprise the helical structures. In another embodiment, the bar arms and the connectors may comprise the helical structures. In further another embodiment, the bar arms, the crowns, and the connectors may comprise the helical structures. In addition, as described above, the helical structures of the bar arms, the crowns, and the connectors may respectively be continuous or discontinuous helical structures; thus, the properties of the stent such as deliverability can be precisely controlled.

Referring to the stent of the present invention mentioned above, in addition to the continuous or discontinuous helical structures applied to the bar arms, the crowns, the connectors, or combinations thereof, the helical structures may also have a single helical structure, a double helical structure, or a combination thereof in order to control the stent properties, such as deliverability.

In the present invention, it should be understood that the term “the bar arms may comprise the helical structures” refers to a portion of the bar arm or to the whole bar arm comprising the helical structure, wherein the helical structure may be a continuous structure or a discontinuous structure, and may also be a single helical structure, a double helical structure, or a combination thereof. A person skilled in the art may adjust the combination thereof as needed, and the present invention is not limited herein. Similarly, in the case that the helical structures are comprised in the bar arms and the connectors (and the crowns), the helical structures may be comprised in a portion of or all of the bar arms and the connectors (and the crowns), may further be a continuous structure or a discontinuous structure, and may also be a single helical structure, a double helical structure, or a combination thereof. A person skilled in the art may adjust the combination thereof as needed, and the present invention is not limited herein.

In addition, the cross-sectional shapes (or the cross-sectional shapes vertical to the strut central axes) of the helical structures and/or the non-helical structures that are comprised in at least one of the bar arms, the crowns, and the connectors may be the shapes described above, such as circular, oval, triangular, rectangular, hexagonal, octagonal, polygonal and airfoil shapes, or any combination thereof. Moreover, in the case that the helical structures comprised in the stent of the present invention are the double helical structures, the cross-sectional shapes of each of the helical structures may be identical or different shapes selected from the shapes described above or any combination thereof. In other words, the double helical structures may be formed by helical structures with different cross-sectional shapes. Furthermore, in the case that at least one of the bar arms, the crowns, and the connectors comprise the helical structures, the cross-sectional shapes of at least one of the bar arms, the crowns, and the connectors are the shapes that are defined by the helical structures. That is, the helical structures of at least one of the bar arms, the crowns, and the connectors may be helical structures with a structure that is one of the group of circular, oval, triangular, rectangular, hexagonal, octagonal, polygonal and airfoil shapes, or any combination thereof.

In the stent of the present invention, at least one of the bar arms, the crowns, and the connectors may have a solid structure, a hollow structure, or a combination thereof. Preferably, at least one of the bar arms, the crowns, and the connectors may comprise the hollow structure for storing drugs. The cross-sectional shape and the hollow structure thereof may be the shapes described above of combinations thereof. In addition, the hollow structures may comprise an interior wall, and a cross-sectional shape of the interior wall may be selected from the group of circular, oval, triangular, rectangular, hexagonal, octagonal, polygonal, and airfoil shapes, or any combination thereof.

In the stent of the present invention, any material in the art suitable for forming the stent comprising the helical structures or having the specific cross-sectional shapes can be used for manufacturing the stent of the present invention, and the present invention is not particularly limited thereto. In a preferred embodiment of the present invention, the stent may be made of a metal, an alloy, a polymer, or any combination thereof. More specifically, the metal may be Ni, Ti, Co, Ta, Cr, Pt, Mg, Fe, an alloy thereof, a stainless steel, or any combination thereof; and the polymer may be a bioresorbable polymer such as poly(L-lactide) (PLLA), polyglycolic acid (PGA), polycaprolactone (PCL), poly(DL-lactide) (PDLLA), polydioxanone (PDS), or any combination thereof. However, the present invention is not limited thereto.

In the stent of the present invention, each of the radially-expandable rings is composed of the bar arms and the crowns, wherein adjacent crowns are connected by the bar arms therebetween. In other words, the bar arms and the crowns may be alternately arranged and connected to one another to form every radially-expandable ring. More preferably, in an exemplary embodiment of the present invention, the bar arms and the crowns may be alternately arranged and connected to one another in a continuous wavy shape to form every radially-expandable ring. Furthermore, the radially-expandable rings comprising the bar arms and the crowns may be closed loops or open loops. In other words, when the radially-expandable rings are closed loops, the independent radially-expandable rings may be arranged along a longitudinal axis and be connected by the connectors disposed between the radially-expandable rings. When each of the radially-expandable rings is an open loop, an endpoint of an opening of one radially-expandable ring is connected to an adjacent endpoint of an opening of another adjacent radially-expandable ring along a longitudinal axis to form a macroscopically helical stent. However, as long as the above-described object of the present invention can be achieved, one having ordinary skill in the art can use any conventional intravascular stent design, and the present invention is not particularly limited thereto.

Furthermore, in the stent of the present invention, as long as the stent with helical structures or specific cross-sectional shapes can be achieved, any preparation methods can be used, and the present invention is not particularly limited. Preferably, in the embodiment of the present invention, the stent of the present invention may be prepared by 3D additive printing.

### BRIEF DESCRIPTION OF THE DRAWINGS

FIG. 1A shows a three-dimensional schematic diagram of the stent 1 according to Example 1 of the present invention;

FIG. 1B shows an enlarged view of part A of the stent 1 according to Example 1 of the present invention;

FIG. 2A shows a three-dimensional schematic diagram of the stent 2 according to Example 2 of the present invention;

FIG. 2B shows an enlarged view of part B of the stent 2 according to Example 2 of the present invention;

FIG. 3A shows a three-dimensional schematic diagram of the stent 3 according to Example 3 of the present invention;

FIG. 3B shows an enlarged view of part C of the stent 3 according to Example 3 of the present invention;

FIG. 4A shows a three-dimensional schematic diagram of the stent 4 according to Example 4 of the present invention;

FIG. 4B shows an enlarged view of part D of the stent 4 according to Example 4 of the present invention;

FIG. 5 shows a cross-sectional view of the stent of Example 5 of the present invention;

FIG. 6 shows a cross-sectional view of the stent of Example 6 of the present invention;

FIG. 7 shows a cross-sectional view of the stent of Example 7 of the present invention; and

FIG. 8 shows a three-dimensional schematic diagram of the stent 8 according to Example 8 of the present invention.

### DETAILED DESCRIPTION OF THE PREFERRED EMBODIMENT

In the following description, numerous specific details are set forth to provide a thorough understanding of embodiments of the present disclosure. However, one having ordinary skill in the art will recognize that embodiments of the disclosure can be practiced without these specific details. In some instances, well-known structures and processes are not described in detail to avoid unnecessarily obscuring embodiments of the present disclosure.

### Example 1

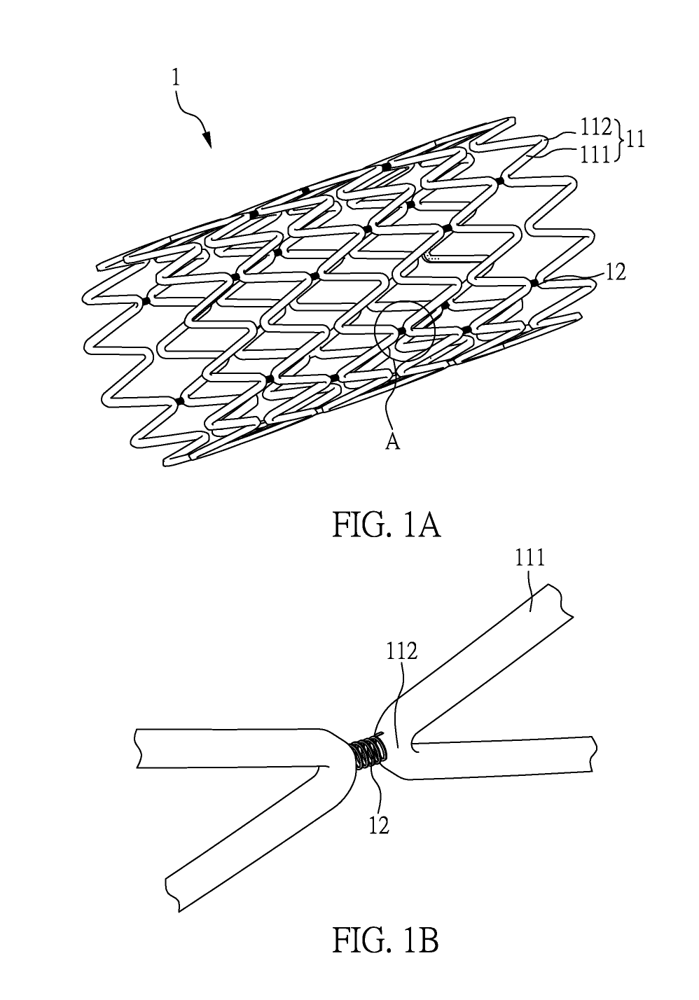

FIG. 1A and 1B respectively show a three-dimensional schematic diagram of the intravascular stent 1 and an enlarged view of its part A according to Example 1 of the present invention. As shown in FIG. 1A, the stent 1 comprises: a plurality of radially-expandable rings 11 arranged along a longitudinal axis, wherein each of the radially-expandable rings 11 respectively comprises a plurality of bar arms 111 and a plurality of crowns 112, and the adjacent crowns 112 are connected by the bar arms 111 therebetween; and a plurality of connectors 12 are disposed between and connecting the radially-expandable rings 11. As shown in FIG. 1B, the connectors 12 comprise helical structures.

In the present example, each of the helical structures is a single helical structure. However, a person skilled in the art can modify the helical structures into double helical structures or a combination thereof if needed, and the present invention is not limited thereto. In the intravascular stent 1 of the present example, the cross-sectional shape of the bar arms 111, the crowns 112, and the connectors 12 is circular, and the bar arms 111, the crowns 112, and the connectors 12 have solid structures. Furthermore, the intravascular stent 1 of the present example may be made of any material, and the present invention is not particularly limited.

Furthermore, in order to realize the above design of applying the helical structures to the connectors, the 3D additive printing manufacturing technique is employed in the present example to prepare the intravascular stent 1. Therefore, the helical structures may be applied to the bar arms, the crowns, and the connectors to improve the stent properties such as deliverability. In the present example, a person skilled in the art can appropriately adjust the parameters of the 3D additive printing in accordance with various characteristics such as the selected material composition, the required processing conditions and so on, but the present invention is not particularly limited and not repeated herein.

### Example 2

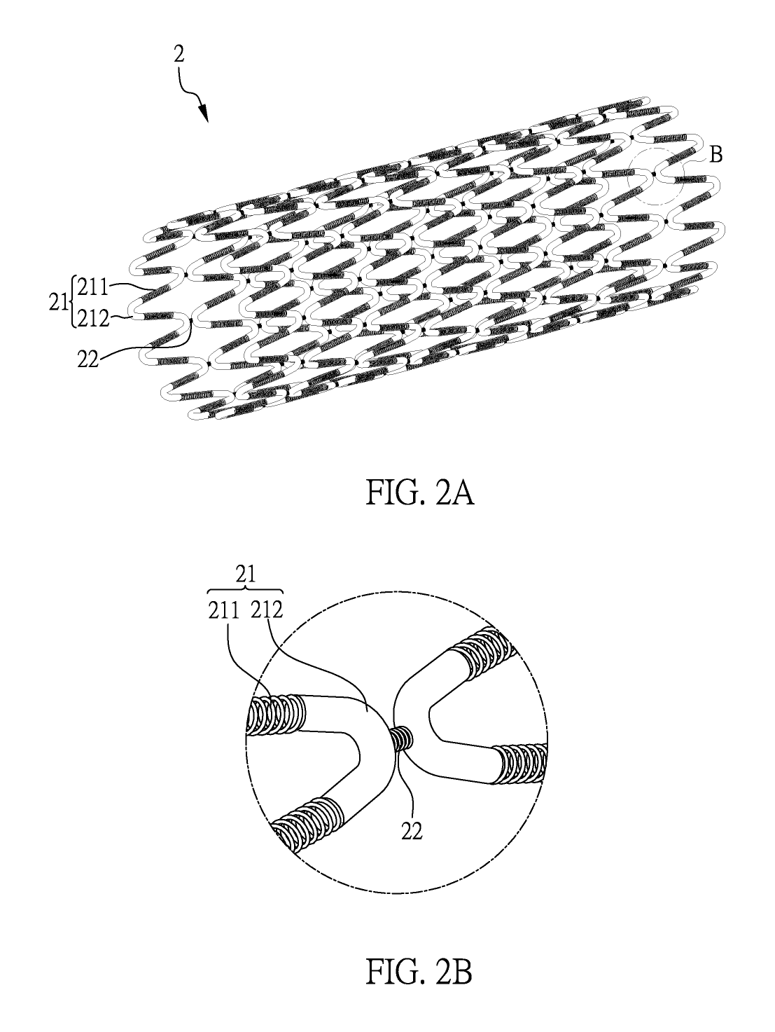

Please refer to FIG. 2A and FIG. 2B, respectively showing a three-dimensional schematic diagram of the stent 2 and an enlarged view of its part B according to Example 2 of the present invention.

Example 2 and Example 1 are substantially the same, except that the bar arms 211 of the stent 2 of Example 2 also comprise helical structures.

Therefore, as shown in FIG. 2A, the stent 2 comprises: a plurality of radially-expandable rings 21 arranged along a longitudinal axis, wherein each of the radially-expandable rings 21 respectively comprises a plurality of bar arms 211 and a plurality of crowns 212, and the adjacent crowns 212 are connected by the bar arms 211 therebetween; and a plurality of connectors 22 are disposed between and connecting the radially-expandable rings 21. As shown in FIG. 2B, the bar arms 211 and the connectors 22 comprise helical structures.

### Example 3

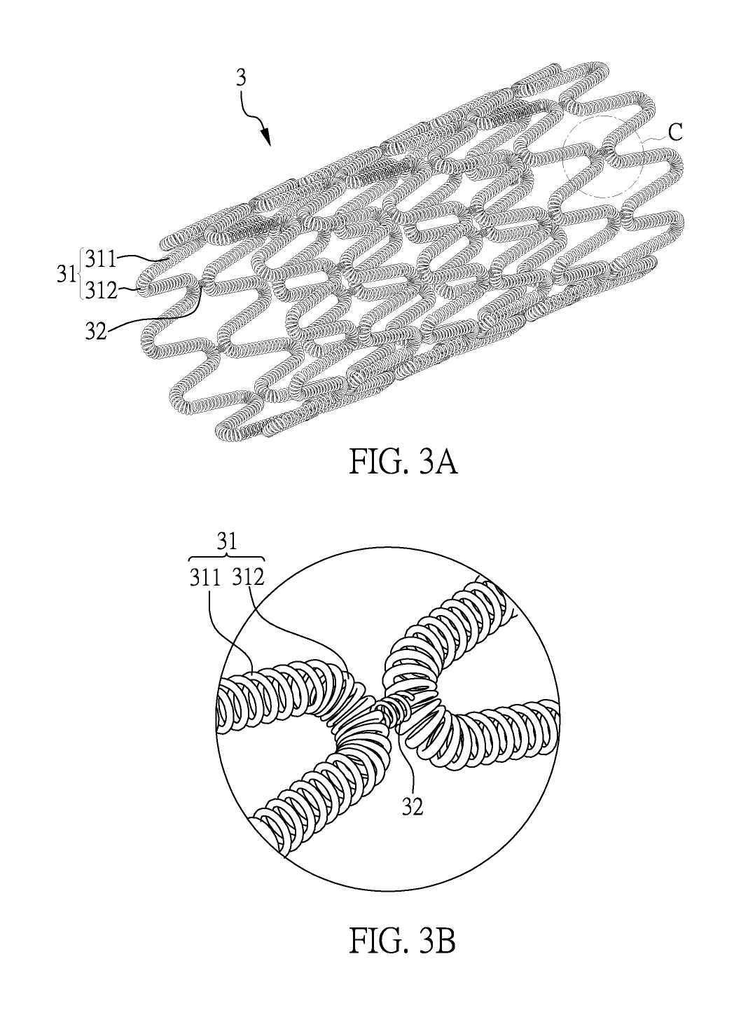

Please refer to FIG. 3A and FIG. 3B, which respectively show a three-dimensional schematic diagram of the stent 3 and an enlarged view of its part C according to Example 3 the present invention. As shown in FIG. 3A, the stent 3 comprises: a plurality of radially-expandable rings 31 arranged along a longitudinal axis, wherein each of the radially-expandable rings 31 respectively comprises a plurality of bar arms 311 and a plurality of crowns 312, and the adjacent crowns 312 are connected by the bar arms 311 therebetween; and a plurality of connectors 32 are disposed between and connecting the radially-expandable rings 31. As shown in FIG. 3B, the bar arms 311, the crowns 312, and the connectors 32 respectively comprise helical structures.

In the present example, the bar arms 311, the crowns 312, and the connectors 32 are composed of helical structures. In addition, the helical structures of the present example are single helical structures. However, a person skilled in the art may replace the single helical structures with the double helical structures or a combination thereof as needed, and the present invention is not limited thereto. In the stent 3 of the present example, the cross-sectional shapes of the bar arms, the crowns, and the connectors are circular, and the bar arms, the crowns, and the connectors respectively have solid structures. Furthermore, the stent 3 of the present example may be prepared with any material, which is not particularly limited in the present invention.

In addition, in order to realize the design for the above mentioned bar arms, crowns, and connectors comprising helical structures, the 3D additive printing manufacturing technique is employed to prepare the stent 3 of the present example. Therefore, the helical structures may be applied to the bar arms, the crowns, and the connectors to achieve the object of improving the stent properties such as deliverability. In the present example, a person skilled in the art may appropriately adjust the parameters of the additive printing in accordance with various characteristics such as the selected material composition and processing conditions, but the present invention is not particularly limited.

### Example 4

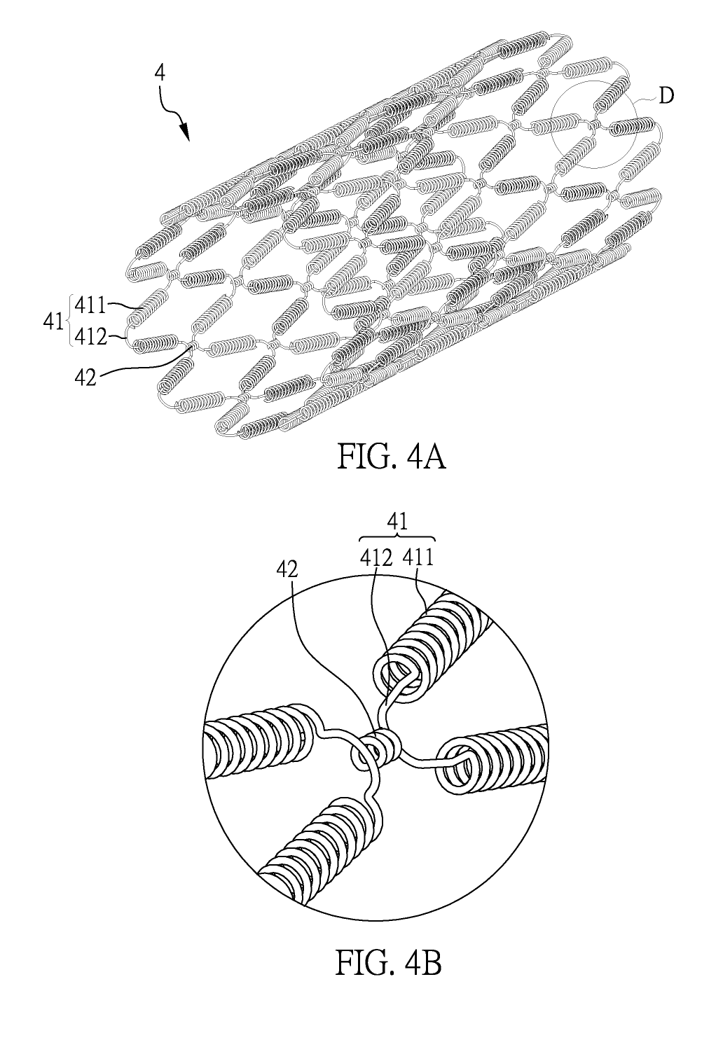

Please refer to FIG. 4A and FIG. 4B, which respectively show a three-dimensional schematic diagram of the stent 4 and an enlarged view of part D of the stent 4 according to the present example. Example 4 and Example 2 are substantially the same, except that the diameter of the cross-sectional shape of the crowns 412 is the same as the diameter of the cross-sectional shape of the helical structures comprised in the bar arms 411 according to the stent 4 of Example 4.

Therefore, as shown in FIG. 4A, the stent 4 comprises: a plurality of radially-expandable rings 41 arranged along a longitudinal axis, wherein each of the radially-expandable rings 41 respectively comprises a plurality of bar arms 411 and a plurality of crowns 412, and the adjacent crowns 412 are connected by the bar arms 411 therebetween; and a plurality of connectors 42 are disposed between and connecting the radially-expandable rings 41. As shown in FIG. 4B, the bar arms 411 and the connectors 42 comprise helical structures.

The manufacturing methods and the material composition used in Example 4 are substantially the same as those used in Example 2, and therefore are not repeated herein.

### Example 5

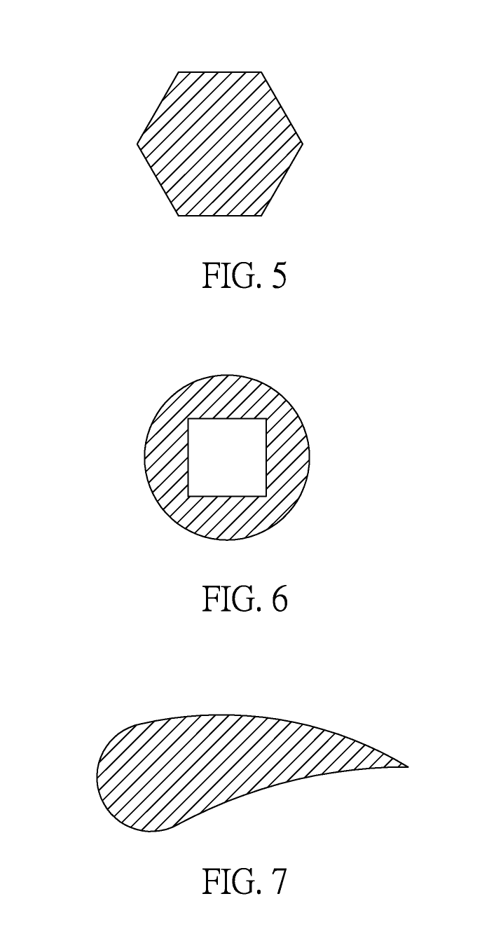

Example 5 and Example 2 are substantially the same, except that the cross-sectional shape is different. Please refer to FIG. 5, which shows a cross-sectional view of the stent strut of Example 5. As shown in FIG. 5, in the stent of Example 5, the cross-sectional shape of the bar arms, the crowns, and the connectors is hexagonal, and the bar arms, the crowns, and the connectors respectively have solid structures.

Similarly, the manufacturing methods and the material composition used in Example 5 are substantially the same as those used in Example 2, and therefore are not repeated herein.

### Example 6

Example 6 and Example 2 are substantially the same, except that the cross-sectional shape is different. Please refer to FIG. 6, which shows the cross-sectional view of the stent strut of Example 6. As shown in FIG. 6, in the stent of Example 6, the cross-sectional shape of the bar arms, the crowns, and the connectors is circular, and the bar arms, the crowns, and the connectors respectively have hollow structures, wherein the cross-sectional shapes of the holes of the hollow structures are rectangular. The holes of the hollow structures may be used as spaces for storing drugs, and the drugs may be released while the stent is implanted.

Similarly, the manufacturing methods and the material composition used in Example 6 are substantially the same as those used in Example 2, and therefore are not repeated herein.

### Example 7

Example 7 and Example 2 are substantially the same, except that the cross-sectional shape is different. Please refer to FIG. 7, which shows the cross-sectional view of the stent strut of Example 7. As shown in FIG. 7, the cross-sectional shape of the bar arms, the crowns, and the connectors is an airfoil shape, and the bar arms, the crowns, and the connectors respectively have solid structures.

Similarly, the manufacturing methods and the material composition used in Example 7 are substantially the same as those used in Example 2, and are not repeated herein.

### Example 8

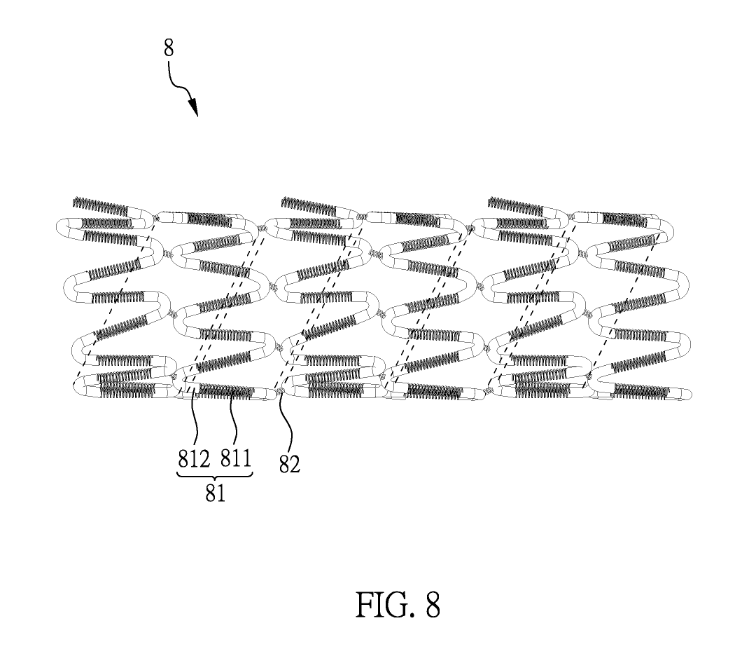

Please refer to FIG. 8, which shows the stent 8 of Example 8. Example 8 and Example 2 are substantially the same, except that the radially-expandable rings of Example 8 are open loops, wherein the endpoint of an opening of one radially-expandable ring is connected to the adjacent endpoint of the opening of the adjacent radially-expandable rings along a longitudinal axis to form a macroscopically helical stent.

Therefore, as shown in FIG. 8, the stent 8 comprises: a plurality of radially-expandable rings 81 arranged along a longitudinal axis, wherein each of the radially-expandable rings 81 comprises a plurality of bar arms 811 and a plurality of crowns 812, and the adjacent crowns 812 are connected by the bar arms 811 therebetween; and a plurality of connectors 82 are disposed between and connecting the radially-expandable rings 81. The bar arms 811 and the connectors 82 comprise helical structures, and the radially-expandable rings 81 are connected to one another and centered along a longitudinal axis to form a helical stent.

The manufacturing methods and the material composition used in Example 8 are substantially the same as those used in Example 2, and are not repeated herein.

It should be understood that these examples are merely illustrative of the present invention; the scope of the invention should not be construed to be defined thereby, and the scope of the present invention will be limited only by the appended claims.

What is claimed is:

1. A stent, comprising:
a plurality of radially-expandable rings arranged along a longitudinal axis, wherein each of the radially-expandable rings comprises a plurality of bar arms and a plurality of crowns, wherein adjacent crowns are connected by the bar arms therebetween; and
a plurality of connectors disposed between the radially-expandable rings for connecting the radially-expandable rings;
wherein a cross-sectional shape of at least one of the bar arms, the crowns, or the connectors is selected from the group of circular, oval, triangular, rectangular, hexagonal, octagonal, polygonal, and airfoil shapes, or any combination thereof.

2. The stent as claimed in claim 1, wherein at least one of the bar arms, the crowns, and the connectors comprises a helical structure.

3. The stent as claimed in claim 2, wherein the connectors comprise the helical structures.

4. The stent as claimed in claim 2, wherein the bar arms comprise the helical structures.

5. The stent as claimed in claim 3, wherein the bar arms and the connectors comprise the helical structures.

6. The stent as claimed in claim 5, wherein the bar arms, the crowns, and the connectors comprise the helical structures.

7. The stent as claimed in claim 2, wherein the helical structure is a single helical structure, a double helical structure, or any combination thereof.

8. The stent as claimed in claim 1, wherein at least one of the bar arms, the crowns, and the connectors has a solid structure, a hollow structure, or any combination thereof.

9. The stent as claimed in claim 1, wherein the stent is made of a metal, an alloy, a polymer, or any combination thereof.

10. The stent as claimed in claim 9, wherein the metal is Ni, Ti, Co, Ta, Cr, Pt, Mg, Fe, an alloy thereof, a stainless steel, or any combination thereof.

11. The stent as claimed in claim 9, wherein the polymer is poly(L-lactide) (PLLA), polyglycolic acid (PGA), polycaprolactone (PCL), poly(DL-lactide) (PDLLA), polydioxanone (PDS), or any combination thereof.

12. The stent as claimed in claim 1, wherein the radially-expandable rings are closed loops or open loops.

13. The stent as claimed in claim 12, wherein when the radially-expandable rings are the open loops, an endpoint of an opening of one radially-expandable ring is connected to an adjacent endpoint of an opening of another adjacent radially-expandable ring.

14. The stent as claimed in claim 1, wherein the stent is prepared by a 3D additive printing method.

15. The stent as claimed in claim 8, wherein the hollow structures comprises an interior wall, and a cross-sectional shape of the interior wall is selected from the group of circular, oval, triangular, rectangular, hexagonal, octagonal, polygonal, and airfoil shapes, or any combination thereof.
